(19) United States
(12) Patent Application Publication
(10) Pub. No.: US 2007/0207118 A1
(43) Pub. Date: Sep. 6, 2007

(54) Surface modification of islet membranes by polymeric grafting methods

(76) Inventors: Young Ro Byun, Kwangsan-ku (KR); Kyung Wook Yang, Jinju (KR); Dong Yun Lee, Kwangmyung (KR)

Correspondence Address: ALAN J. HOWARTH, P.O. BOX 1909, SANDY, UT, 84091-1909, US

(21) Appl. No.: 11/656,893

(22) Filed: Jan. 22, 2007

Related U.S. Application Data
(63) Continuation of application No. 10/398,794, filed on Apr. 8, 2003.

(30) Foreign Application Priority Data
Nov. 3, 2000 (KR) PCT/KR00/01254
Oct. 24, 2000 (KR) 2000-62667

(51) Int. Cl. C12N 11/02; A61K 35/39; A61K 9/00

(52) U.S. Cl. 424/78.17; 424/556; 528/354

(57) ABSTRACT

The present invention provides surface modification methods of pancreatic islet membranes by polymeric grafting. Particularly, the present invention provides the surface modification methods that hydrophilic polymer chain is grafted onto collagen membranes of the pancreatic islets by various polymeric grafting methods instead of encapsulation of the pancreatic islets. Since the surface modification methods of the present invention minimize immunorejection without islet damage in islet transplantation, extend efficiency and survival time of the pancreatic islets by maintaining a high diffusion rate of oxygen and nutrient and reduce total volume of the pancreatic islets required for islet transplantation, they can be effectively used for transplantation of the pancreatic islets.

### FIELD OF THE INVENTION

The present invention relates to surface modification of pancreatic islet membranes using a polymer. Particularly, the present invention relates to surface modification resulted from combining a polymeric chain like polyethylene glycol (PEG) to the surface of glycogen membrane of pancreatic islet using various polymeric grafting methods instead of encapsulation of pancreatic islet.

### BACKGROUND

Diabetes is characterized with high blood sugar caused by absolute or relative lack of insulin and various disorders carried with it. There are two types of diabetes. Type 1, insulin-dependent diabetes, is caused by a massive destruction of insulin secreting β-cells of islet and type 2, insulin-independent diabetes, also relates to defect of insulin secretion against glucose. However, the exact mechanism of such malfunctioning of insulin secretion has not been explained yet.

The fundamental treatment for type I diabetes is islet transplantation. Unlike the insulin-injection method, the islet transplantation helps secretion or insulin or glucagon responding to a change or blood sugar to keep a constant blood-sugar level, and secretion of enzyme in need, which makes it the only permanent cure for the late insulin-dependent diabetic patients. In case of auto-transplantation, pancreatic islet is less destructed, but the chance of getting it is very limited and the immune response after transplantation has been a problem to solve.

The immune response herein means the destruction of transplanted pancreatic islet by immune cells. To solve this problem, it is necessary to develop a pancreatic islet, which does not cause immune response. To overcome these matters, other ways such as allo-transplantation and xeno-transplantation have been developed. The transplantation of a pancreatic islet of a pig into human is an example of xeno-transplantation, which has merit of enough supplying of pancreatic islets but has demerit of serious refuse action against them, leading in destruction of pancreatic islet within 2 weeks. Thus, the development of a novel pancreatic islet without carrying immune response is the key point of the pancreatic islet transplantation techniques.

In order to inhibit the immune response after islet transplantation, various devices have been proposed. For examples, an intravascular device, microencapsulation, macrocapsulation have been developed. These methods are very effective in suppressing immune response but they still have fundamental problems such as the problems of viability of islet and the volume of a device and islet transplant to be.

The viability of islet relates to the supply of oxygen and nutrients necessary for the survival of the islets. In case of encapsulation of islet, owing to the rather long distance between islet and surrounding blood vessels, the oxygen and nutrients are not supplied fully, thus the islet cannot respond to a change of blood-sugar sensitively, resulting in failure of the effective secretion of insulin. The islet of a normal pancreas has micro blood vessels, so that the distance between cells in pancreatic islet and blood vessels is just about ten micrometer. However, once the islet is encapsulated, the distance becomes hundreds of micrometers, which cause the decrease of viability of transplanted islet and the insulin secretion.

Another big problem of islet transplantation using encapsulation is concerning the volume of the device encapsulating the pancreatic islets. The normal volume of pancreatic islet of human is about 10 ml, but when the islet is encapsulated with microcapsule, the size of it enlarges by more than 10-fold, which put the transplantation of needed islet in trouble.

To perform a successful transplantation of pancreatic islet, it is important to protect pancreatic islet from immune response. Two different types of immunoisolation devices were developed in order to substitute the harmed pancreas. One is intravascular device and the other is extravascular device. The extravascular device is divided into macrocapsule device and microcapsule device. The intravascular device is supposed to be transplanted into subcutaneous tissue or into peritoneal cavity.

(1) Intravascular Device

Pancreas is an organ with a very delicate blood vessel structure, and pancreatic islet is supplied with oxygen and nutrients through the blood vessels spread in pancreas. Therefore, it is required to develop a device having a balanced blood vessel system for an effective transplantation. Generally, the intravascular device consists of membrane tubes, which surround pancreatic islet. Blood flows through the membrane tubes carrying oxygen as nutrients and eliminating wastes secreted from pancreatic islet. This membrane covering pancreatic islet prevents immune cells from infiltrating into intravascular device.

However, the above device has a problem to cause thrombosis when being transplanted. Such induced blood clots inhibit blood flowing through the membrane tubes of intravascular device. For that reason, blood flow speed decreases and the supply of oxygen and nutrients becomes difficult and so does the elimination of wastes. The membrane is important for immune moderation, but at the same time it causes problems such as thrombosis, inhibition of controlling the concentration of blood sugar, etc. In addition, the intravascular device gives another difficulty in surgical operation for transplantation.

(2) Extravascular Device

(2-1) Microcapsule

The most preferred method for microencapsulation of pancreatic islet is to envelop pancreatic islet using hydrophilic alginate gel following polymer coatings to form a semipermeable membrane on the surface of pancreatic islet. To make a proper solution to be used for encapsulation of islet, alginic acid is injected in the water solution containing divalent cations. Microcapsulation of pancreatic islet generally increases vitality of cells comparing with other immune moderating devices. Comparing to macrocapsule, the membrane of microcapsule surrounding islet is thinner, which makes the supply of oxygen and nutrients easy and fast. Microcapsule also has higher diffusion rate and quicker response to the change or glucose concentration owing to the short diffusing distance from the outside of microcapsule to the islet inside capsule.

Those characteristics make microcapsulation of islet very useful method, in separating the pancreatic islet away from immune system. This method, however, still has problems. The biggest problem is that the aggregation of islet inside microcapsule prohibits supplying oxygen and nutrients to the center cells of aggregated islet. Another problem is the difficulty in removing microcapsule. Microcapsule should be removed by additional surgery in order to prevent continuing immune response caused by long-term necrosis of islet inside microcapsule

(2-2) Macrocapsule

Macrocapsulation is to envelop a large number of cells or tissues in order to separate immune system. The membrane covering the bulk of pancreatic islet is similar to inner blood system. The above membrane prevents phagocytic cells such as lymphocytes and macrophages from penetrating, so that immune response is restrained. The membrane, meanwhile, permits oxygen and nutrients like glucose to be spread harmoniously. The membrane is in the shape of hollow fiber, sheet or disc, which allows it to be applied to a variety of cells and tissues.

The biggest merit of macrocapsule is its easiness in surgical operation on account of that all pancreatic islets are gathered into the center space, and its easiness with less danger when being removed. Meanwhile, the above membrane is so thin that it is easy to be torn. Once the membrane is torn, fragments left around it causes immune response, which is of course dangerous. Thus, it is important for the membrane to have strong resistance against any stress in order to prevent a mechanical default. Another problem of macrocapsule is that the supply of oxygen and nutrients is limited and wastes are accumulated in the capsule.

Despite the fact that new blood vessels are well generated in it, the essential nutrients and oxygen are not supplied enough to the center-located pancreatic islet since the macrocapsule surrounds outside the whole islet. And the following coagulation of islet inside the limited space leads to the acceleration of the necrosis of pancreatic islet. Even if macrocapsulated pancreatic islet is transplanted for the treatment of diabetes mellitus, the islet cannot always secrete the normal level of insulin. Thus, in order to supplement low level of insulin secretion, transplanted pancreatic islet should be enlarged in its size, which is another problem.

To overcome the above problems, instead of performing polymer coating, the present inventors have developed a method that uses polymeric chains like polyethylene glycol (referred as “PEG” hereinafter) anchored to the surface of pancreatic islet. This method prevents the destruction of islet by immune response and also enables the full supply of oxygen and nutrients is possible owing to the space made from polymeric chain, resulting the efficiency and survival time of pancreatic islet is increased.

PEG is a macromolecule, which is widely used in the field of biomedical science and pharmacology as well. PEG chain has high flexibility and exists as hydrated status since its interfacial energy is minimized under water-soluble condition. PEG chain attached on the islet surface is compressed by approaching of other elements or proteins, which results in the loss of entropy. To prevent the entropy loss, PEG should keep its elastic property to push out the approaching elements. Along with the elastic property, its limited capacity contributes to the prohibition of the approach of other elements or proteins. Those characteristics of PEG can be used in variety, especially in giving biocompatibility onto macromolecule surfaces.

PEG can combine with proteins, medicinal substances or liposome. Such PEG combined proteins or medicinal substances have been reported to avoid phagocytosis, by which their circulating time and half-life could be last long. Thus, combination of molecules having protein or medicinal activity with synthetic macromolecules provides great advantages when being applied to experiments both in vivo and in vitro. The covalent bond of synthetic macromolecules with physiologically active molecules can change the surface property of molecules and their solubility; that is to say, it can increase solubility against water or organic solvents. Besides, the combination also increases biocompatibility but decreases immune response, resulting in increase of stability in the living body as well as decrease of clearance caused by intestine system, kidney, spleen or liver. Thus, binding peptide to macromolecule increases stability in a solution and protects the intrinsic surface property of peptide effectively, resulting in prevention of unspecific protein adhesion.

The U.S. Pat. No. 4,179,337 referred to the peptide-macromolecule complex which was generated by combining peptide and polypeptide with PEG weighing 500-20,000 in its molecule weight or water-soluble polymer that kept biological activity and at the same time decreased antigenicity and anti-immunity. And, U.S. Pat. No. 4,301,144 described that when hemoglobin was combined with PEG or water-soluble polymer, its oxygen carrying ability is increased.

Abuchowski and his colleagues (Abuchowski, et al., Cancer Biochem. Bioohys., 7, 175-186, 1984) have proved that many proteins combining with PEG showed increased half-life and decreased immunity in blood-plasma, and Davis and his colleagues (Davis, et al., Lancet, 2, 281-283, 1981) have proved that when uricase was combined with PEG, its half-life in a body was increased and the side-effect of uric acid metabolism was decreased.

On the bases of those results, it has been confirmed that combining PEG with biologically active peptides or proteins increased half-life in vivo, increased solubility, and decreased immune response.

The most preferred method to combine PEG with polypeptide is to react biologically activated PEG with polypeptide amino residue.

Lysine group and N-terminal are the examples of polypeptide amino residue and the way to activate PEG is as follows: replacing one of the two hydroxy group of PEG with methyl ether group, and combining the other hydroxy group with functional group having electrophile (Abuchowski, A. and Davis, F. F., 1981, in Enzymes as Drugs, Holsengberg, J. and Roberts, J., eds). The examples of the activated macromolecules are PEG-N-hydroxysuccinimide-activated esters having amide bond, PEG-epoxide and PEG-tresylate having alkyl bond, PEG-carbonyl imidazole and PEG-nitrophenyl carbonates having urethane bond, PEG-aldehyde having Schiff's base, etc.

Lysine groups exist in polypeptide randomly, so that if PEG which responses to an amino group is used, PEG is combined unspecifically with any of those groups, resulting in generation of uneven mixture. Therefore, recent studies have focused on the way to combine PEG with specific target groups such as cysteine group, oligo sugar, hydroxy group, arginine group, etc, in order to prepare an even PEG corporate body.

PEG inducers responding specifically to a cysteine group of polypeptide are PEG-vinyl sulfone, PEG-iodoacetamide, PEG-maleimide, PEG-orthopyridyl disulfide, etc, of which PEG-maleimide is the most widely used. PEG-vinyl sulfone has the best stability in a water solution and PEG-orthopyridyl disulfide can be reversibly resolved in a body owing to its disulfide bond. Interleukin-3 and interleukin-2 are the examples of peptides using the above inducers.

PEG-hydrazide is an example of PEG inducer responding specifically to an oligo sugar of polypeptide which constructs relatively stable hydrazone bond by responding to aldehyde containing materials. PEG-hydrazide is characteristically combined with the sugar part of glycoprotein, and for that reason, it is preferably used for a specific target binding.

PEG-isocyanate is a PEG inducer responding specifically to a hydroxy group of polypeptide. If it is necessary to combine PEG with arginine group of polypeptide, a PEG inducer containing phenylglyoxal which responses well to a guanidino group is used.

As explained above, when a macromolecule like PEG is combined with a physiologically active molecule, it not only changes the surface property of a molecule and its solubility but also increases biocompatibility and its stability in vivo, while decreasing immune response. Using these characteristics, the present inventors have anchored a macromolecule, PEG, onto the surface of pancreatic islet membrane, leading to the improvement of the surface of membrane. The present invention has been accomplished by proving that the pancreatic islet, having improved its membrane surface by the novel method of the present invention, can minimize the immune response caused by transplantation and maximize the efficiency and life time of the islet by a smooth supply of oxygen and nutrients as well as avoiding the problem of volume when being manufactured.

### SUMMARY OF THE INVENTION

The objective of this invention is to provide a method to modify the surface of pancreatic islet membrane by grafting a polymeric chain like PEG onto the surface of the islet, resulting in the prevention of destruction of the islet by immune response and increase of the efficiency and life time of islet by sufficient supply of oxygen and nutrients owing to the enough room spared by the macromolecule chain.

### BRIEF DESCRIPTION OF THE DRAWINGS

FIG. 1 is a diagram showing the procedure of substituting end group (—OH) of monomethoxy polyethylene glycol (referred as “mmPEG” hereinafter) with —COOH group.

FIG. 2 is a diagram showing the activation procedure of mPEG whose end group has been substituted with —COOH group.

FIG. 3a is a photograph showing the morphological change of pancreatic islet by the grafting of pancreatic islet with PEG according to the reaction time, and this figure is specifically representing the morphological change at 0 hour after reaction began.

FIG. 3b is a photograph showing the morphological change of pancreatic islet by the grafting of pancreatic islet with PEG according to the reaction time, and this figure is specifically representing the morphological change at 1 hour after reaction began.

FIG. 3c is a photograph showing the morphological change of pancreatic islet by the grafting of pancreatic islet with PEG according to the reaction time, and this figure is specifically representing the morphological change at 2 hour after reaction began.

FIG. 3d is a photograph showing the morphological change of pancreatic islet by the grafting of pancreatic islet with PEG according to the reaction time, and this figure is specifically representing the morphological change at 3 hour after reaction began.

FIG. 3e is a photograph showing the morphological change of pancreatic islet by the grafting of pancreatic islet with PEG according to the reaction time, and this figure is specifically representing the morphological change at 4 hour after reaction began.

FIG. 4a is a photograph showing the confocal scanning image of PEG grafted pancreatic islet 1 hour after reaction.

FIG. 4b is a photograph showing the confocal scanning image of PEG grafted pancreatic islet 2 hours after reaction.

FIG. 4c is a photograph showing the confocal scanning image of PEG grafted pancreatic islet 3 hours after reaction.

FIG. 4d is a photograph showing the confocal scanning image of PEG grafted pancreatic islet 4 hours after reaction.

FIG. 5a is a photograph showing the morphological change of pancreatic islet according to the repetitive grafting reaction with PEG, and this figure is specifically representing the control pancreatic islet.

F FIG. 5b is a photograph showing the morphological change of pancreatic islet according to the repetitive grafting reaction with PEG, and this figure is specifically representing the shape of pancreatic islet after the first PEG grafting reaction.

FIG. 5c is a photograph showing the morphological change of pancreatic islet according to the repetitive grafting reaction with PEG, and this figure is specifically representing the shape of pancreatic islet after the second PEG grafting reaction.

FIG. 5d is a photograph showing the morphological change of pancreatic islet according to the repetitive grafting reaction with PEG, and this figure is specifically representing the shape of pancreatic islet are the third PEG grafting reaction.

FIG. 6a is a photograph showing the confocal scanning image of pancreatic islet according to the repetitive grafting reaction with PEG, and this figure is specifically representing the shape of pancreatic islet after the first PEG grafting reaction.

FIG. 6b is a photograph showing the confocal scanning image of pancreatic islet according to the repetitive grafting reaction with PEG, and this figure is specifically representing the shape of pancreatic islet after the second PEG grafting reaction.

FIG. 6c is a photograph showing the confocal scanning image of pancreatic islet according to the repetitive grafting reaction with PEG, and this figure is specifically representing the shape of pancreatic islet after the third PEG grafting reaction.

FIG. 7 is a graph showing the result of 18 days observation upon insulin secretion reaction against sugar in PEG grafted pancreatic islet.

FIG. 8 is a graph showing the result of 28 days observation upon insulin secretion reaction against sugar in repetitively PEG grafted pancreatic islet.

FIG. 9 is a graph showing the insulin secretion reaction curve against refluxed sugar stimulus in PEG grafted pancreatic islet.

FIG. 10 is a graph showing the insulin-secretion curve against sugar stimulus in pancreatic islet repetitively grafted with PEG.

FIG. 11 is a graph showing the result of 60 days observation upon concentration change of blood sugar of a diabetic animal.

FIG. 12a is a graph showing the result of observation upon concentration change of blood sugar of a diabetic animal after being transplanted with PEG grafted pancreatic islet.

FIG. 12b is a graph showing the result of observation upon weight change of a diabetic animal after being transplanted with PEG grafted pancreatic islet.

FIG. 13 is a photograph showing the result of H-E staining over the kidney membrane (14 days after transplantation) of a control pancreatic islet transplanted animal.

FIG. 14 is a photograph showing the result of H-E staining over the kidney membrane (100 days after transplantation) of a PEG grafted pancreatic islet transplanted animal.

FIG. 15 is a picture showing the microencapsulation of PEG-grafted pancreatic islet.

FIG. 16 is a picture showing the microencapsulation of PEG-grafted pancreatic islet (covered with AN69 membrane).

FIG. 17a is a picture showing the pancreatic islet combined with mPEG having various molecular weights.

FIG. 17b is a picture showing the PEG-grafted pancreatic islet after performing PEG amplification using polyacrylic acid and polyvinyl alcohol.

FIG. 17c is a picture showing the PEG-grafted pancreatic islet after sulfonating PEG.

### DETAILED DESCRIPTION OF PREFERRED EMBODIMENTS

The present invention provides surface modification methods of pancreatic islet membranes that polymer chain is grafted onto surfaces of the pancreatic islet membranes by polymeric grafting.

The surface modification methods of the present invention is to grafting chemically synthetic macromolecules, biopolymers or their derivatives and complexes onto collagen membrane of pancreatic islet, which leads to the restraining of immune response caused by pancreatic islet-transplantation.

The surface modification method of pancreatic islet membrane consists of the steps as below:
1) activating step of end groups of polymers; and
2) grafting step of the above activated polymers onto the surface of pancreatic islet membrane.

As polymers in the step 1, synthetic polymers, biopolymers or their derivatives and complexes could be used, and especially, hydrophilic polymers which have outstanding biocompatibility and below 90 degree contact angle with water are preferred. For examples, poly[acrylic acid], poly[acrylate], poly[acrylamide], poly[vinylester], poly[vinyl alcohol], polystryene, polyoxide, cellulose, starch, polysaccharide, polyelectrolyte, poly[1-nitropropylene], poly [N-vinyl pyrrolidone], poly[vinyl amide], polyethylene glycol (PEG), and their derivatives or complexes are preferred. In addition, hydrophobic polyurethane and poly[dimethyl siloxanel] as a siliconic polymer could be used as well.

In the preferred embodiments of the present invention, a hydrophilic polymer PEG was used. Since PEG has big hydrodynamic volume and great ability of holding water molecules, PEG combined with collagen membrane of pancreatic islet forms water molecule membrane on the pancreatic islet membrane, resultantly, it prevents from being recognized as antibody by immune cells. PEG also inhibits immune response against transplanted pancreatic islet by pushing phagocytes out with its thermodynamic function when phagocytes are penetrated into pancreatic islet. The preferable molecular weight of PEG is from 100 to 1,000,000 dalton, and monomethoxy polyethylene glycol, succinimide of PEG propionic acid, succinimide of PEG butanoic acid, branched PEG-NHS, PEG succinimidyl succinate, succinimide or carboxymethylated PEG, benzotriazole carbonate of PEG, PEG-glycidyl ether, PEG-oxycarbonylimidazole, PEG-nitrophenyl carbonates, PEG-aldehyde and their analogues or complexes can be used.

Besides, various polymer derivatives attached functional group at the end or side chain of polymer can be used as well. Sulfone group attached polymers or heparin combined polymers are the examples. Amine group, hydroxyl group, carboxyl group, sulfone group and their compounds can be used as a functional group. Meanwhile, unlike the above synthetic polymers, biopolymers having anti-thrombus activity also can be used. As examples, polysaccharide such as heparin or prostaglandin (PGI) can be used. And, recombinant heparin, heparin derivative and heparin analogues, which have similar characteristics to heparin can be used instead of the above heparin.

In the step 1), carboxylation has been accomplished for substituting the end group (—OH) of the polymer with the end group (—COOH) which shows better reaction. The way of activating the carboxylated polymer is to substitute hydroxy group of the one side of the polymer with methyl ether groups, and bind a functional group to the other side hydroxy group of the polymer.

In the step 2), in order to bind polymers to pancreatic islet, functional groups of islet such as hydroxy group, amine group, carboxyl group have been grafted to the end or side chain of polymers having corresponding functional groups. DCC (N,N-dicyclohexylcarbodiimide, Y-K Lee et al., Thromb. Res., 92, 149-156, 1998), EDC(N-(3-dimethylaminopropyl)-N′-ethyl carbodiimide hydrochloride, D. H. Rich and J. Singh, “The carbodiimide method” in The Peptides: Vol. 1, Academic Press, New York, 1979, pp. 241-261), and photoreaction can be used for grafting of polymers with pancreatic islet membranes. The polymers grafted to the islet membranes by covalent bond can be lasted stably after transplantation.

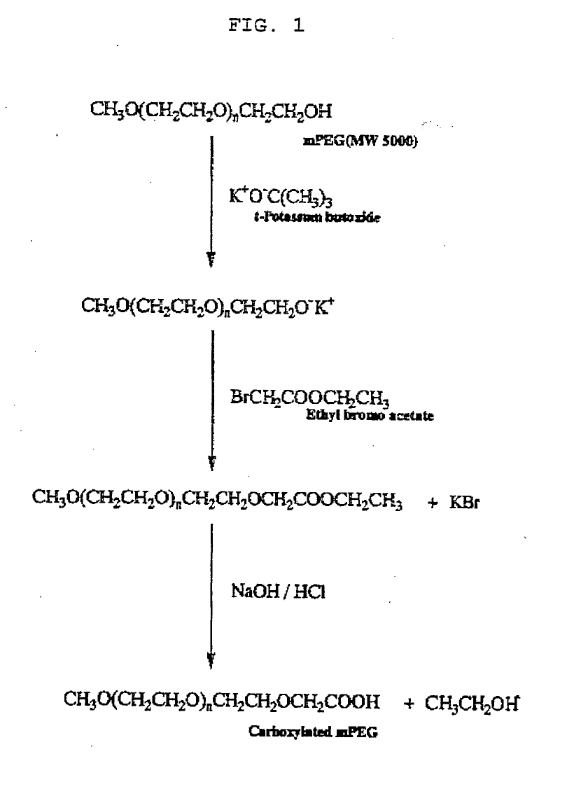

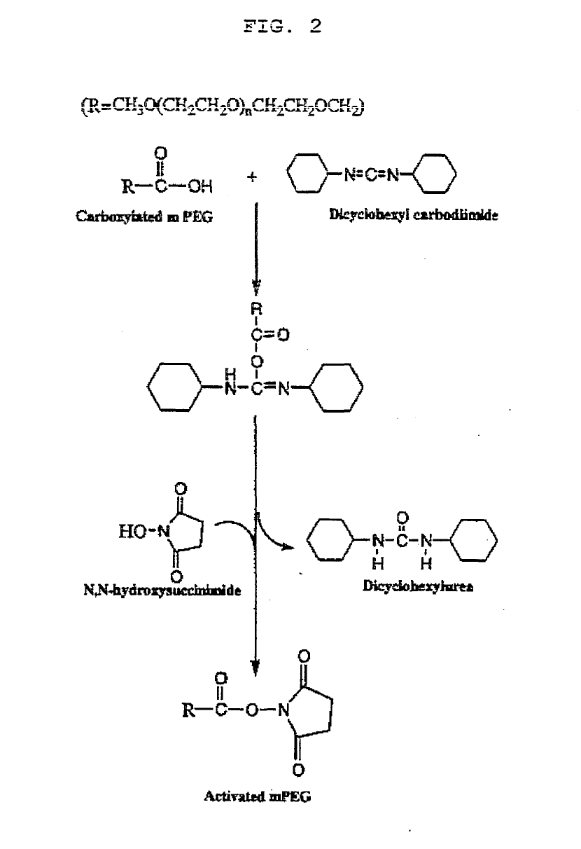

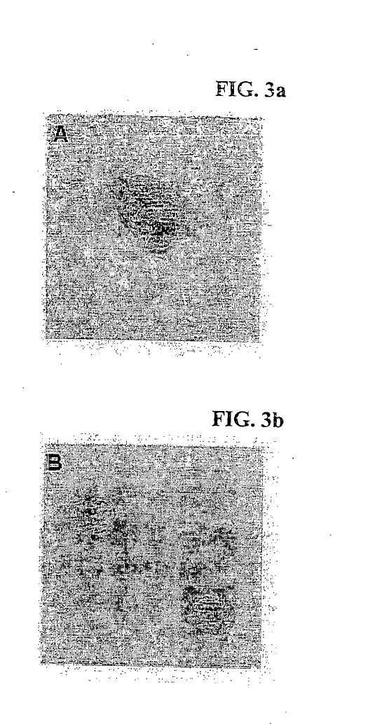

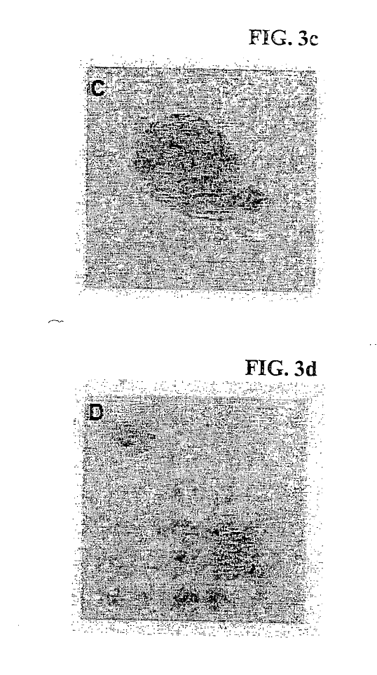

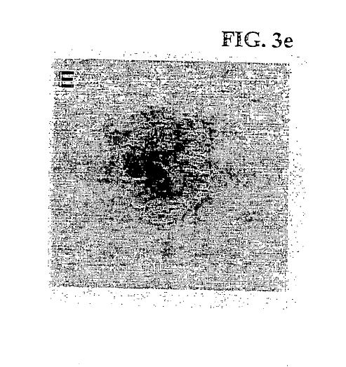

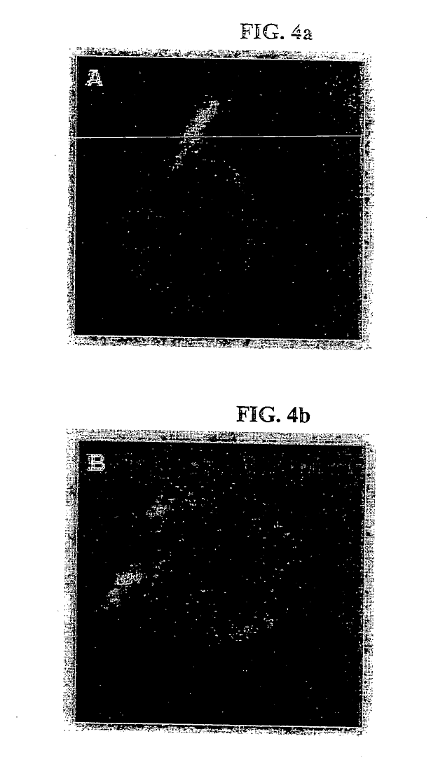

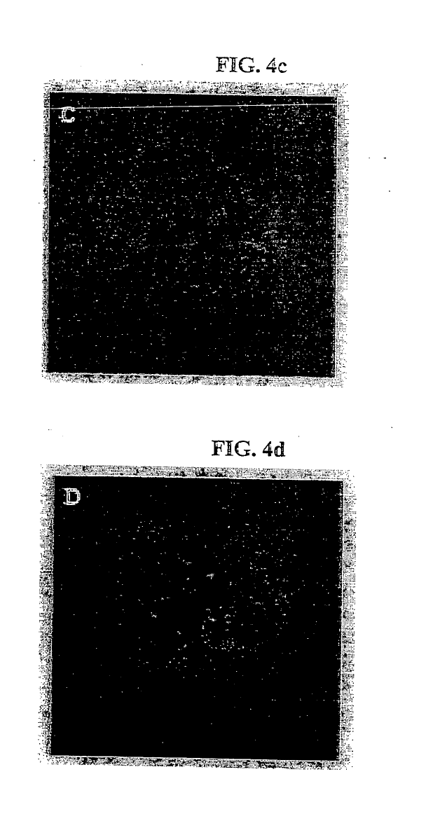

In the preferred embodiments, the present inventors have grafted PEG to the surface of pancreatic islet with various methods. Firstly, the end group (—OH) of PEG (molecular weight=5000) has been substituted with the very reactive end group (—COOH) (see FIG. 1), and then, the activated above polymer has been grafted to the prepared rat pancreatic islet (see FIG. 2). For the best restraining of immune response, repetitive reaction has been accomplished to maximize the density of grafted PEG. Successive PEG grafting to the sue ace has been confirmed by using confocal scanning microscopy. PEG has been grafted effectively to the surface of pancreatic islet and some PEG has penetrated inside the membrane (see FIGS. 3a-3e, 4a-4-d, 5a-5d and 6a-6c). This phenomenon can be controlled by regulating reaction time of PEG grafting to the pancreatic islet membrane.

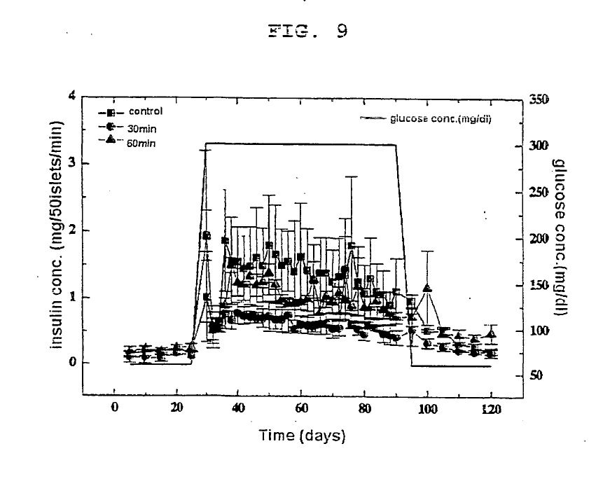

Through in vitro test using static culture and perifusion method, pancreatic islet whose surface was modified by PEG has been verified to be able to secret same amount of insulin as untreated islet (see FIGS. 7, 8, 9 and 10).

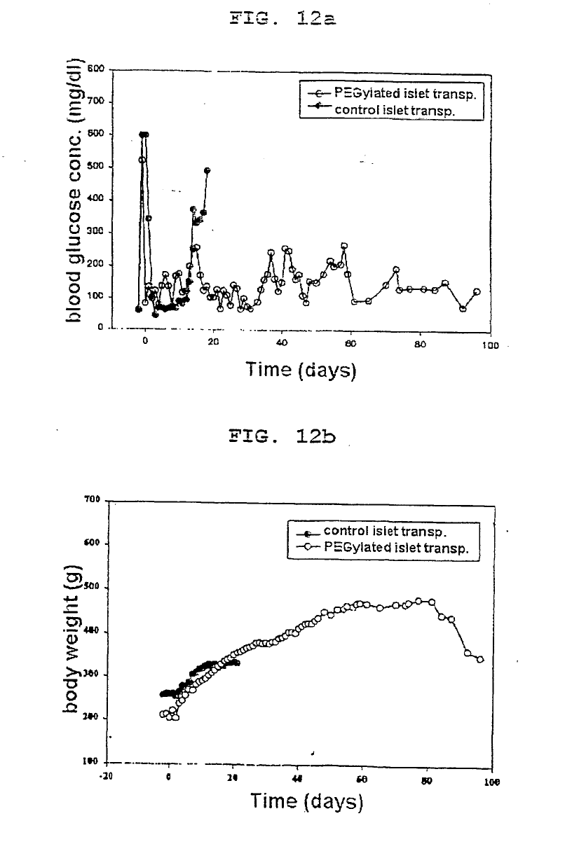

Meanwhile, through in vivo animal test, PEG grafted pancreatic islet was transplanted onto the kidney membrane of a diabetic animal, and then his blood-sugar level and weight were measured to see if the transplanted pancreatic islet works normally. As a result, the test animal transplanted with PEG grafted pancreatic islet kept normal blood-sugar level for a long while and showed gradual increase in his weight. On the contrary, the test animal transplanted with control pancreatic islet kept normal blood-sugar level just for 2 weeks and from then on his blood-sugar level went high, that is to say, went back to diabetic condition again. Therefore, it is confirmed that the PEG grafted pancreatic islet of present invention survives longer and secrets insulin more properly than the control group (see FIGS. 11 and 12).

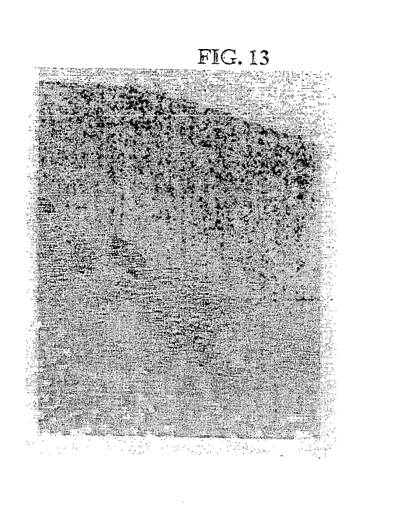

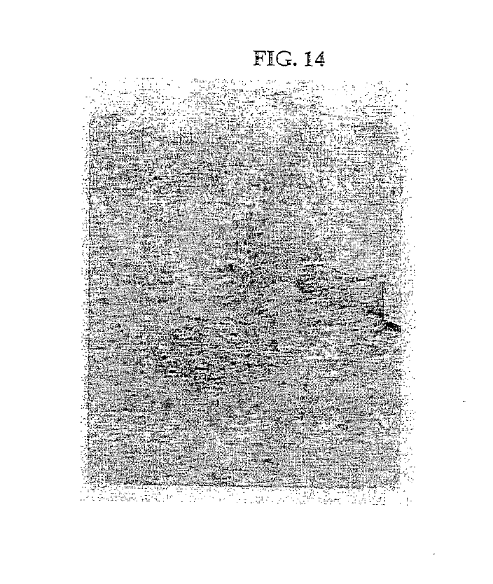

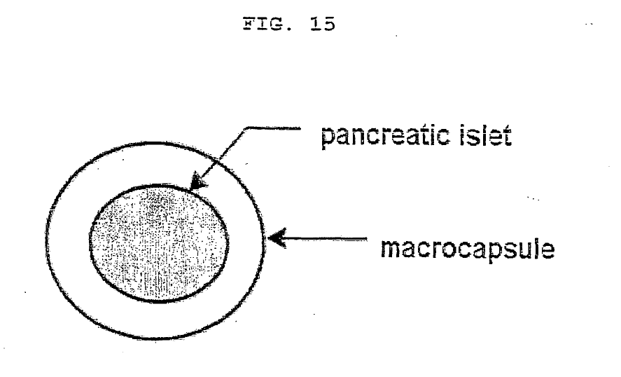

Kidney membranes of every test animals were isolated to examine their tissues since those are the spots where transplantation would be done. As a result, T-lymphocytes around PEG grafted pancreatic islet were decreased drastically and no lymphocytes that were detected penetrated inside pancreatic islet (see FIGS. 13 and 14).

Conclusively, it is confirmed that PEG grafted pancreatic islet moderates immune response and still functions normally.

Additionally, pancreatic islet of the present invention whose surface has modified by polymeric grafting methods can be transplanted without any special device, which makes it not be troubled by large volume caused by clinically devised pancreatic islet on the basis of immune separating methods. After being modified, the pancreatic islet shows no change of volume and shows excellent vitality and reaction of pancreatic islet cells by enough supply of oxygen and nutrients from blood vessels around when being transplanted.

Moreover, surface modification method of pancreatic islet membrane by using polymers or the present invention can be used in variety in addition to the case of pancreatic islet transplantation and have strong points to restrain aggregation of pancreatic islet itself. In other words, since the surface of pancreatic islet membrane is modified by hydrophilic polymers, pancreatic islet does not curdled each other when being used in microcapsulation or macrocapsulation, leading to high chance of its survive.

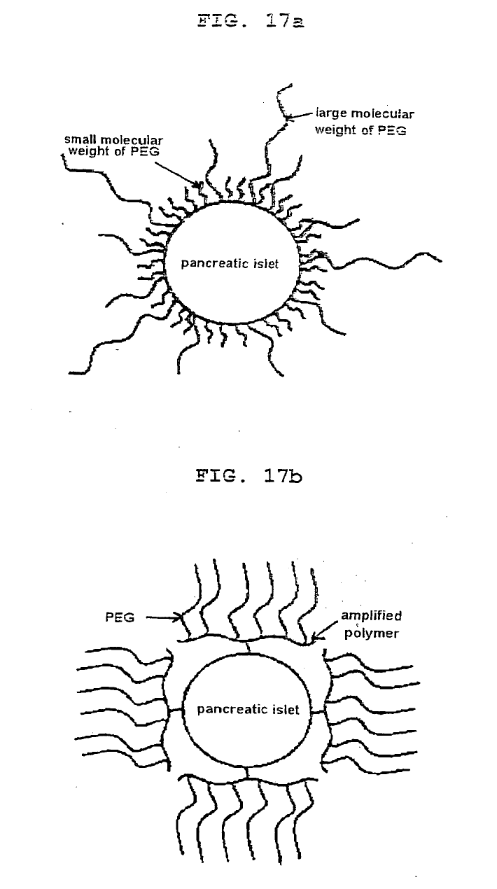

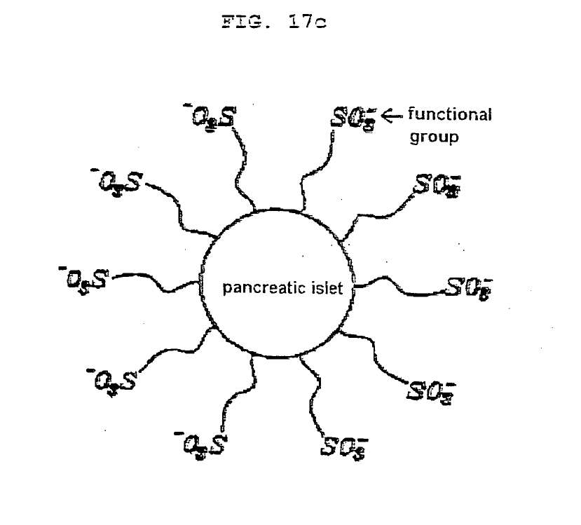

In order to combine mPEG having various molecular weights repeatedly to the surface of pancreatic islet membrane, the present inventors reacted activated MW 5000 PEG, MW 2000 PEG and MW 200 PEG repeatedly to the surface of pancreatic islet membrane (see FIG. 17a). Pancreatic islet grafted with various PEG having different molecular weight have increased density, resulting in minimized immune response and better supply of oxygen and nutrients compared with the pancreatic islet modified with simple PEG having same molecular weight.

And, in order to maximize the effect of PEG grafting to the surface of pancreatic islet membrane, the present inventors have grafted PEG to two types of functional groups, hydroxyl group and amine group, existing thereon. More precisely, PEG was activated by DCC method to be grafted to amine group existing on the surface of pancreatic islet membrane, and NCO group was attached to the end of functional group of PEG bay treating 4,4′-diphenylmethane diisocyanate (MDI) or toluene diisocyanate (TDI) in order to graft PEG to hydroxyl group.

The present inventors also have performed PEG amplification using polyacrylic acid (PAA) and polyvinyl alcohol (PVA) (see FIG. 17b). Specifically, polyacrylic acid (MW=5000) was treated with methylene chloride, N-hydrosucciinimide and 1,3-dicyclohexyl carbodiimide, and activated PAA obtained thereafter was used for PEG amplification on the pancreatic islet membrane. Polyvinyl alcohol, meanwhile, was treated with sodium periodate (NaIO4), resulting in the substitution of all hydroxyl group (—OH group) existing in PVA with aldehyde group (—CH group), and the activated PVA obtained thereafter was used for PEG amplification.

The present inventors has further induced sulfonation of PEG for the grafting of functional PEG to the pancreatic islet membrane in cases where PEG has amino group and hydroxyl group at each ends (HO-PEG-NH2) (see FIG. 17c). For the sulfonation, amino group of PEG was substituted with sulfonyl group by loading propane sultone into PEG using tetrahydrofuran (THF) as a solvent. Otherwise, toluene is added to PEG in order to substitute hydroxyl group (—OH group) so PEG with carboxyl group (—COOH group), and then the PEG is warmed up while circulating pure Ar at high temperature. When phase separation is occurred by the condensation reaction of PEG and toluene, unclear water has been removed and potassium butoxide and ethyl-3-bromopionate are added instead. PEG obtained therefrom has sulfone group and carboxyl group at each ends, thus carboxyl group at one end is grafted to the pancreatic islet membrane and sulfonyl group at the other end is electrolyzed having repulsive negative electricity, by which a transplanted pancreatic islet is able to prohibit immune materials approaching and keep itself stable for a long while.

In case where PEG has carboxyl groups at both ends (HOOC-PEG-COOH), methylene chloride, N-hydrosuccinimide and 1,3-dicyclohexylcarbodiimide need to be added for the activation. The activated carboxyl group of one end of PEG obtained therefrom is grafted to the pancreatic islet membrane and the other side carboxyl group is electrolyzed having repulsive negative electricity, by which a transplanted pancreatic islet is able to prohibit immune materials approaching and keep itself stable for a long while.

### EXAMPLES

Practical and presently preferred embodiments of the present invention are illustrative as shown in the following Examples.

However, it will be appreciated that those skilled in the art, on consideration of this disclosure, may make modifications and improvements within the spirit and scope of the present invention.

### Example 1

### PEG Grafting on Pancreatic Islet Membrane

<1-1> Carboxylation Step of mPEG

45 g of mPEG-5000 was solved with 500 ml water in 3-neck flask and added with toluene anhydrous. While keeping the temperature at 120° C., pure Ar was continuously circulated. mPEG and toluene mixed solution was broiled and condensation had been occurred. As phase separation was seen, unclear water was removed and the temperature was lowered to room temperature. Potassium butoxide was added to the above mixed solution and the mixture was reacted at 70° C. for 24 hours. After that, the temperature was lowered to room temperature again and ethyl-3-bromopropionate was added and let the mixture react at room temperature for one day. The mixture was filtered to remove potassium bromide (KBr) and precipitated by adding ether. The precipitated mixture was then stored at freezer for 2 or 3 hours and filtered again and finally dried. Dried PEG was mixed with 200 to 300 ml of 1 M NaOH and stirred for 2 hours. 2 hours later, 15 g of NaCl was added thereto and its pH was regulated until it was reached pH 3 by using 6 M HCl. Chloroform was added thereto and phase separation was followed. Then only chloroform layer was removed and MgSO₄ was added to remove remaining moisture. The obtained mixture was filtered and precipitated in ether, after which it was filtered again and dried under the reduced pressure for one day, and finally the reaction product was obtained (FIG. 1).

<1-2> Activation Step of Carboxylated mPEG

mPEG was activated according to the following steps to react against amine group existing on the surface of pancreatic islet. Firstly, mPEG-COOH was dissolved by adding methylene chloride in 2-neck flask. Secondly, N-hydrosuccinimide was added thereto and waited until the solution was dissolved completely. Thereafter, the solution was put in an ice bath and added with 1,3-dicyclohexylcarbodiimide, which was suspended for one day before being filtered. Methylene chloride was then evaporated and 50-100 ml of benzene was added. 6 hours later, the solution was filtered again. The solution was precipitated in ether and filtered again, from which the reaction product was finally obtained (FIG. 2).

<1-3> Separation of Pancreatic Islet

A SD rat (Korea Experimental Animal Center, Korea) in the weight of 250-300 g, which fasted from one day before islet separation, was anesthetized with subcutaneous injection of ketamine and xylazine mixture. After its abdomen was opened, HBSS (Hanks' Balance Salt Solution: 0.14 g CaCl₂, 0.4 g KCl, 0.06 g KH₂PO₄, 0.098 g MgSO₄, 8.0 g NaCl, 0.048 g Na₂HPO₄, 1.0 g Glucose, NaHCO₃ 0.35 g, 1% penicillin-streptomycin [10 ml]) was injected through bile duct in order to swell the pancreas. Right after the injection, the swelled pancreas was pulled out. The separated pancreas was cut into pieces with scissors and washed several times to remove fat. 5 mg of collagenase per 1 g of tissue were dissolved in 2 ml of HBSS in 37° C. water bath and the solution was shaken to resolve pancreas properly. Iced HBSS was added thereto in order to prevent over-resolving. The resolved pancreas tissues were filtered and washed several times. Supernatants were removed by centrifugation at 1500 rpm for 5 minutes and 27, 23, 20 and 11% of ficoll density gradients were made, followed by a centrifugation at 2200 rpm for 23 minutes. Islets gathered from layers between 27% and 23% and between 23% and 20% were washed and then, transferred onto culture plates containing RPMI-1640 culture media, and cultured while being supplied with 5% CO2 at 37° C. Culture media was exchanged every other day and pancreatic islet was finally separated therefrom.

<1-4> Grafting Step of Activated mPEG onto Pancreatic Islet Membrane

The separated pancreatic islet obtained in the above example <1-3> was cultured in RPMI-1640 medium for 3 days and then grafted with PEG of example <1-2>. Pancreatic islet washed with HBSS to remove remaining culture medium in order for PEG to be grafted well on the pancreatic islet. 15 ml of HBSS buffer solution in which 23 mg of activated PEG was dissolved was loaded to pancreatic islet, and pancreatic islet and PEG were reacted for 0.5, 1, 2, 3, 4 hour laps of time in incubator (5% CO₂, 95% O₂, 37° C.). The pancreatic islet washed with culture medium to prevent unnecessary further-going reaction after PEGylation. In order to measure the reacted amount of PEG on the surface of pancreatic islet, FITC-PEG marked with fluorescent material was used for the experiment instead of activated PEG. Total amount of reacted PEG was measured by calculating FITC-PEG grafted onto the surface of pancreatic islet with confocal scanning microscopy.

As seen in FIG. 3a to 3e, it was confirmed through the observation with optical microscopy on the changes of shapes of pancreatic islet with the lapse of reaction time that the shape of pancreatic islet was kept stable without any change when PEGylation took 2 hours, whereas the shape of pancreatic islet membrane was changed when PEGylation was going over 4 hours. And also, through the observation on the confocal scanning image of pancreatic islet reacted against FITC-PEG according to each reaction time, it was confirmed that PEG was grafted on the pancreatic islet membrane when the PEGylation kept going over 1 hour and as reaction time was lengthened, PEG tended to penetrate inside pancreatic islet (FIG. 4a to 4d).

### Example 2

### mPEG Grafting onto the Pancreatic Islet Membrane by Repetitive PEGylation

On the basis of the result of the above <example 1>, proper PEGylation time was decided 1 hour and PEGylation was performed once a day repeatedly for 3 days. The morphological change of pancreatic islet membrane was observed with electron microscopy according to each reaction time while PEGylation was performed repeatedly and the density of PEG grafted on the surface of pancreatic islet was confirmed to be increased by repetitive PEGylation of FITC-PEG on the surface of pancreatic islet.

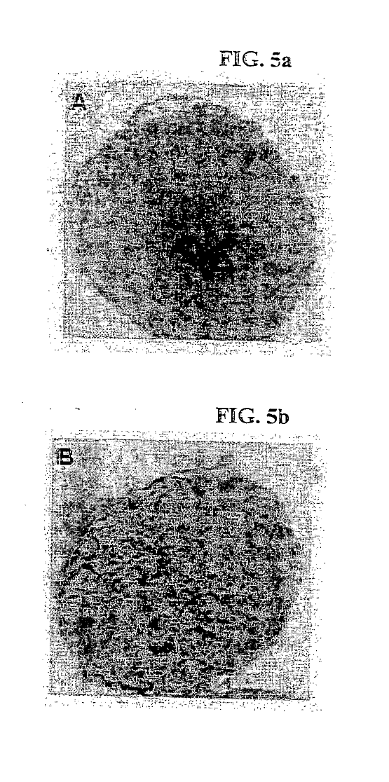

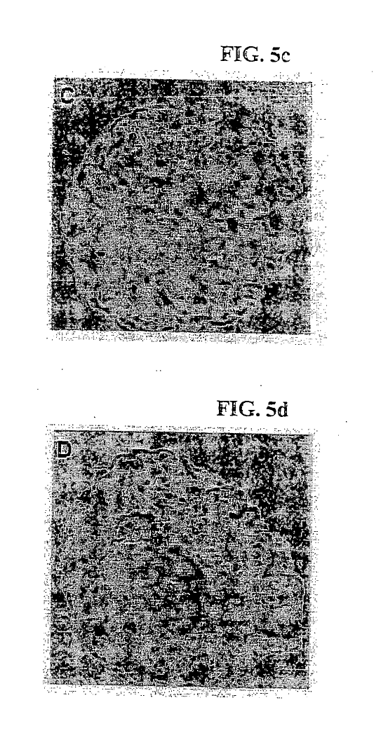

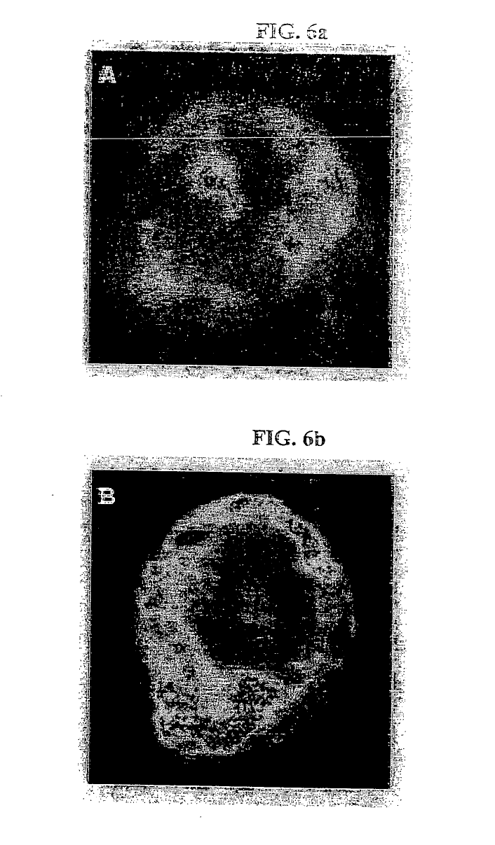

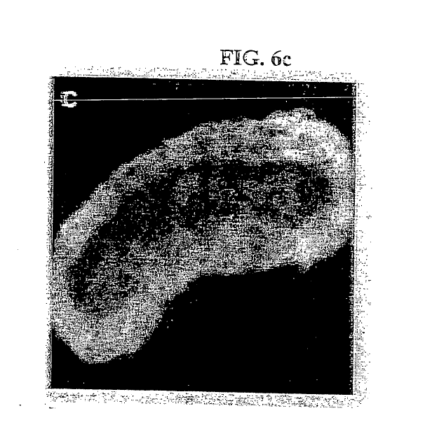

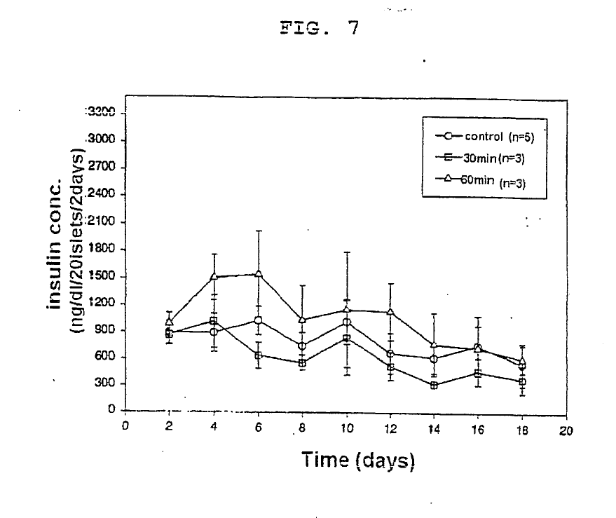

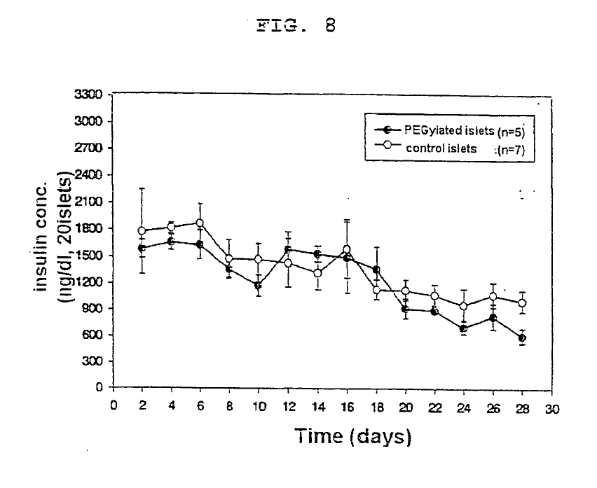

As seen in FIGS. 5a and 5d, repetitive PEGylation did not change the shape of pancreatic islet. It was also confirmed through the observation of confocal scanning image of pancreatic islet onto which PEGylation was performed repeatedly that PEG was clearly grafted onto the pancreatic islet membrane. In addition, the more PEGylation repeated, the higher the density of PEG grafted onto the pancreatic islet membrane went (FIG. 6 to 6c)

### Example 3

### Grafting of mPEGs Having Different Molecular Weights onto Pancreatic Islet Membrane by Repetitive PEGylation

In order to graft various mPEGs having different molecular weights onto pancreatic islet membrane, the present inventors have performed PEGylation repeatedly once a day for 3 days, just like the above <example 2>. At first, separated pancreatic islet obtained in <1-3> washed twice with HBSS buffer solution and 15 ml HBSS in which 23 mg of activated PEG was dissolved was added, followed by 1 hour reaction in incubator. And then, the above solution washed twice with RPMI-1640 culture medium and cultured for one day adding the same culture medium. On the second day, the pancreatic islet washed twice with HBSS buffer solution and 15 ml of HBSS in which 23 mg of activated PEG having 2000 molecular weight was dissolved was added thereto for 1 hour reaction in the incubator. The pancreatic islet washed twice again with RPMI-1640 culture medium and added with the same medium for culture thereafter. On the third day, the pancreatic islet washed twice with HBSS buffer solution and then, 15 ml of HBSS buffer solution in which 23 mg of activated PEG having 600 molecular weight was dissolved was added, followed by on hour incubation. The pancreatic islet washed twice again with RPMI-1640 culture medium and thereafter the same medium was added for culture.

### Example 4

### Grafting of PEG with Various Functional Groups of Pancreatic Islet Membrane

In order to get the most effective grafting of PEG onto pancreatic islet membrane, the present inventors have grafted PEG with two functional groups, which exist hydroxyl group and amine group on the pancreatic islet membrane.

PEGylation with amine group was performed as follows after PEG was activated by the same DCC method as the above <example 1>. In order to graft PEG with hydroxyl group on pancreatic islet membrane, NCO group was attached to the end of functional group of PEG by treating it with 4,4′-diphenyl methane diisocyanate (MDI) or toluene diisocyanate (TDI). After NCO group was attached thereto, the PEGylation was performed. More precisely, 5 g of mPEG having 5000 molecular weight was dissolved in toluene anhydrous solution and MDI was added thereto until the mole concentration rate between hydroxyl group of PEG and NCO group existing on MDI or TDI became 1:5. Ether was added into the above reaction product to be precipitated. The reaction product was filtered and dried under reduced pressure or a day. And then, PEG-NCO was obtained as final reaction product. Meanwhile, the separated pancreatic islet obtained in <1-3> washed with HBSS buffer solution and 15 ml of HBSS buffer solution in which 23 mg of activated PEG was dissolved was added thereto and activated for 1 hour. Thereafter, the pancreatic islet washed twice with RPMI-1640 culture medium and 15 nm of HBSS buffer solution in which 23 mg of PEG-NCO was dissolved was added thereto, leading to the reaction with the pancreatic islet membrane. The pancreatic islet was washed twice again with RPMI-1640 culture medium and the same culture medium was added thereto for further culture.

### Example 5

### PEG Amplification on Pancreatic Islet Membrane

<5-1> PEG Amplification on Pancreatic Islet Membrane Using Polyacrylic Acid (PAA)

Methylene chloride was added to 50 g of polyacrylic acid (PAA, MW=5000), and the PAA was dissolved. N-hydrosucciinimide was added to the above solution. After complete dissolution, 1,3-dicyclohexylcarbodiimide was added thereto in an ice water bath and filtered one day later. Methylene chloride was evaporated from the above filtered solution and 50 ml of benzene was added thereto. 6 hours later, the solution was filtered again. The obtained filtered solution was precipitated in ether and filtered again, after which activated PAA was finally obtained.

In order to amplify the prepared activated PAA on pancreatic islet membrane, pancreatic islet washed twice with HBSS buffer solution at first and 15 ant of HBSS buffer solution in which 25 mg of activated PAA was dissolved was added to the pancreatic islet, which was reacted in incubator for one hour. An hour later, the pancreatic islet was washed twice again with HBSS buffer solution and then 15 ml of HBSS buffer solution in which 50 g of methoxy-PEG-NH2 (MW=5000) was dissolved was added, which was reacted in incubator for one hour again. The pancreatic islet washed twice with RPMI-1640 medium and then the same culture medium was added for further culture.

<5-2> PEG Amplification onto Pancreatic Islet Membrane Using Polyvinyl Alcohol

50 g of polyvinyl alcohol (PVA, MW=5000) was reacted with sodium periodate (NaIO4), from which hydroxyl group (—OH group) existing in PVA was substituted with aldehyde group (—CH group), resulting in the activation of PVA. 15 ml of HBSS buffer solution in which 30 mg of activated PVA was dissolved was added to the pancreatic islet which washed twice with HBSS buffer solution. The solution was cultured in an incubator for an hour. Using HBSS buffer, the pancreatic islet combined with activated PVA washed, and 15 ml of HBSS buffer containing 50 mg of methoxy-PEG-NH₂ was added thereto, and cultured in an incubator for an hour. The pancreatic islet washed twice with RPMI-1640 again and the same culture medium was added thereto for further culturing.

### Example 6

### Functional PEGylation onto Pancreatic Islet Membrane

<6-1> Sulfonation of HO-PEG-NH2 and its Grafting onto Pancreatic Islet Membrane

10% Propane sultone was loaded to 10% HO-PEG-NH2, which was reacted at 5° C. for 5 hours. Tetrahydrofuran (THF) was used as a solvent for the reaction. The reactant was precipitated in THF, filtered and washed with cold THF solvent. Through such reaction, amino group of HO-PEG-NH2 was substituted with sulfonyl group. In order to exchange the rest hydroxyl group (—OH group) of HO-PEG-NH2 for carboxyl group (—COOH group), 40 g of the above reactant was added into 450 ml of toluene to be dissolved. Keeping temperature at 120° C., the vessel containing PEG was warmed up in hot water with circulating pure Ar. As toluene solution mixed with mPEG was boiling, phase separation occurred by condensation reaction. At that moment, unclear water was removed and temperature was lowered. Potassium butoxide was added to the above mixture, which was reacted at 70° C. for 24 hours. After dropping the temperature to the room temperature, ethyl-3-bromopionate was added for one more day reaction. The mixture was filtered to strain KBr out, after which ether was added thereto for precipitation. The precipitated solution was stored at freezer for 2-3 hours and then filtered again and dried. 200-300 ml of 1 M NaOH was added to the prepared dried PEG for 2 hour stirring followed by adding 15 g of NaCl, and the pH of the solution was regulated up to pH 3 using 6 M HCl. Chloroform was added thereto. Upon chase separation occurring, only chloroform layer was removed. Thereafter, MgSO₄ was added to remove remaining moisture. The above solution was filtered and precipitated in ether. The solution was filtered again later and dried under the reduced pressure, resulting in obtaining final reactant.

The above reactant was added to methylene chloride to be dissolved and N-hydrosucciniimide was added thereto. After complete dissolution, 1,3-dicyclohexyl carbodiimide was added in an ice bath and the solution was going under reaction for one day before filtering. Methylene chloride was evaporated from the above filtered solution and then 50 ml of benzene was added. 6 hours later, the solution was filtered again. The filtered solution was precipitated in ether and the final reactant was obtained by repeated filtering. The final product was used for the grafting with pancreatic islet membrane.

Pancreatic islet washed twice with HBSS buffer solution and the HBSS buffer solution having 30 mg of above reactant was added thereto, which was incubated for one hour. It washed twice with RPMI-1640 and the same culture medium was added for further culturing. Through the above process, one end of PEG was grafted onto the pancreatic islet membrane and sulfone group or the other end of PEG was electrolyzed having a negative charge. Owing to the repulsive force of negative charge of the sulfone group, various immune materials were kept apart, resulting in the protection of transplanted pancreatic islet for a long while.

<6-2> Activation of HOOC-PEG-COOH and its Grafting onto Pancreatic Islet Membrane

50 g of PEG having two carboxyl groups (—COOH group) was added to methylene chloride and dissolved. N-hydrosucciniimide was added thereto. After complete dissolution, 1,3-dicyclohexylcarbodiimide was added thereto in an ice bath with the amount of enough mol to activate one carboxyl group and the solution were reacted for one day before filtering. Methylene chloride was evaporated from the above filtered solution and then 50 ml of benzene was added. 6 hours later, the solution was filtered again. The obtained solution was precipitated in ether and filtered again, leading to obtaining final reactant.

15 ml of HBSS buffer solution containing 23 mg of the above reactant was added to pancreatic islet washed with HBSS buffer solution, which was cultured in an incubator for one hour. Thereafter, it washed twice with RPMI-1640 culture medium and the same medium was added for further culturing. By the above process, the activated carboxyl group of one end of PEG was grafted onto pancreatic islet membrane and the carboxyl group of the other end of PEG was electrolyzed having a negative charge. Owing to the repulsive force of negative charge of sulfone group, various immune materials were kept apart, resulting in the protection of pancreatic islet for a long while.

### Example 7

### In Vitro Viability of PEG Grafted Pancreatic Islet

<7-1> In Vitro Static Culture

It order to measure the viability of PEG grafted pancreatic islet in vitro, the present inventors have observed its insulin secretion while culturing the PEG grafted pancreatic islet for a long time. To prevent aggregation of islet, 20 PEG grafted pancreatic islet was loaded on culture plate having inserts in which gelatin sponge was put. M199 (Gibco BRL Co., 5.5 mM glucose, 2 mM sodium pyruvate, 6 mM HEPES, 10% FBS, 1% penicillin/streptomycin) was used as culture medium. The culture medium was exchanged every other day and in the mean time samples were gathered. The same experiment was repeatedly performed with PEG grafted pancreatic islet. Insulin secretion was measured from the samples picked every other day using RIA method (insulin radioimmunoassay, Linco Research Inc.).

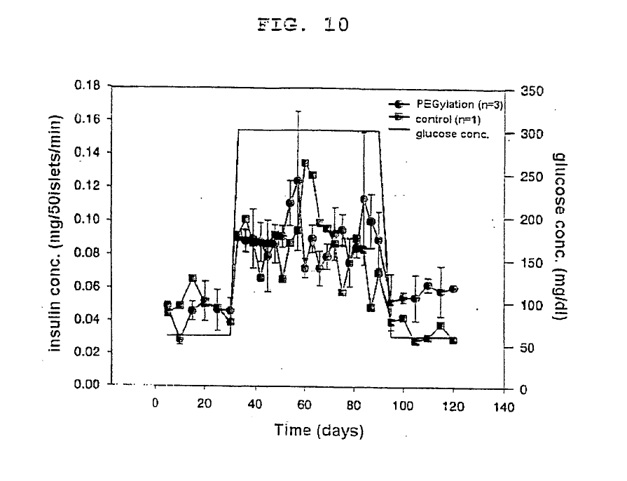

FIG. 7 is showing secretion response curves of insulin against sugar of PEG grafted pancreatic islet and control (PEG ungrafted) pancreatic islet with the lapse of time for 18 days. As seen in FIG. 7, even if PEG was grafted to pancreatic islet, the insulin secreting function of pancreatic islet was not decreased. FIG. 8 is showing secretion response curve of insulin against sugar detected for 4 weeks in the pancreatic islet repeatedly grafted with PEG. This figure also shows that the insulin secreting function of pancreatic islet was not changed even if PEG was grafted to islet repeatedly.

<7-2> In Vitro Perfusion Test

In order to confirm the viability of PEG grafted pancreatic islet under the stimulation of sugar, the present inventors have observed the insulin secretion of pancreatic islet against different concentrations of sugar perfused for a short term. PEG grafted pancreatic islet and control (PEG ungrafted) pancreatic islet, 50 each, were inserted in islet chamber of perfusion tester and through which KRBB culture medium (3.469 g NaCl, 0.175 g KCl, 0.160 g MgSO₄7HO, 0.0816 g KH₂PO₄, 0.1054 g CaCl₃, 1.05 g NaHCO₃, 1.19 g HEPES, 0.025 g BSA, 1 L water) including different concentrations of sugar were perfused while 5% CO₂ was being supplied at 37° C. At first, culture medium with 60 mg/dl of sugar concentration was perfused for 30 minutes and samples were picked every 5 minutes. Then, the sugar concentration of the culture medium was rapidly increased to 300 mg/dl and the medium was perfused again while samples were picked every 3 minutes this time. Lastly, the sugar concentration was down to 60 mg/dl and the medium was perfused while samples were picked every 5 minutes. The same experiment was done with pancreatic islet repeatedly grafted with PEG. The insulin concentrations of all the above examples were measured by using RIA.

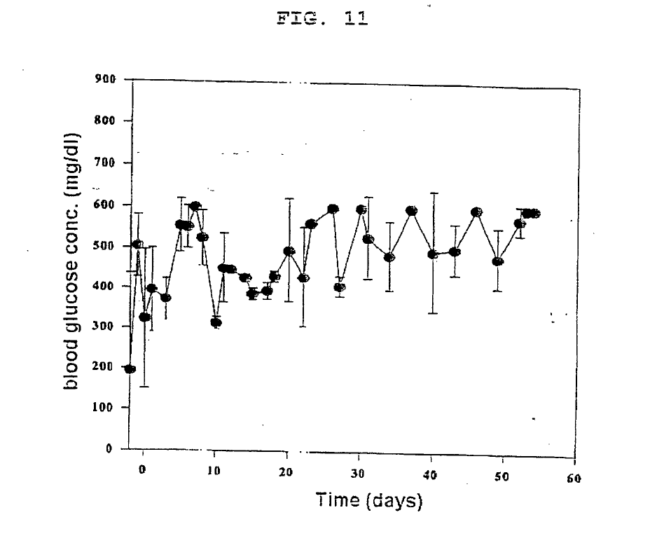

FIG. 9 is showing insulin secretion curves against perfused sugar with the lapse of reaction time for both PEG grafted pancreatic islet and control (ungrafted) islet. As seen in FIG. 9, both pancreatic islets secreted insulin normally against the stimulation of sugar. FIG. 10 is showing insulin secretion curve of pancreatic islet grafted with PEG repeatedly against perfused sugar and it was confirmed as seen in this figure that the islet also secreted insulin normally against the stimulation of sugar.

### Example 8

### Transplantation of PEG Crafted Pancreatic Islet, In Vivo

<8-1> Transplantation of PEG-Grafted Pancreatic Islet

60 mg/kg of streptozotocin was injected to SD rats in the weight of 200-250 g to cause diabetes. Every animal whose blood-sugar concentration reached 400 mg/dl was regarded as diabetic and selected for using as recipients for the pancreatic islet transplantation. FIG. 11 is showing the changes of blood-sugar concentration of chosen animals for 60 days. Those diabetic animals kept the blood-sugar levels as 400-600 mg/dl. 2000 PEG grafted pancreatic islets and 1000 control islets were transplanted to the kidney membranes of those diabetic animals, after which the changes of blood-sugar and the weight of those animals were observed.

FIG. 12 is showing the changes of weight and blood-sugar concentration of diabetic animals after transplantation of PEG grafted pancreatic islet. The animals transplanted with control pancreatic islet showed increased blood-sugar level over 500 mg/dl, 2 weeks after transplantation, however the animals transplanted with PEG grafted pancreatic islet kept normal level of blood-sugar and showed normal increase of weight until 100 days after transplantation.

<8-2 Histology

Kidney of the animal transplanted with PEG grafted pancreatic islet was picked out in order to perform biopsy of both the kidney and the transplanted islet. The kidney was fixed with 10% formalin solution and sample tissues were dipped in paraffin, which were later cut into 5 μm thick pieces using microtome. The prepared pieces were fixed on slide glass. The pieces were dipped in xylene solution this time to remove paraffin and H&E staining was performed. As a result, the cell nucleus was dyed dark blue and the cytoplasm was dyed red. Immuno staining was also done to confirm whether the taken kidney and the transplanted islet had insulin. The staining process was same as that of the above H&E staining, and LSAB kit (DAKO Co.) was used as a dyeing agent.

FIG. 13 is showing the H-E staining result of kidney membrane of an animal 14 days after being transplanted with the control pancreatic islet. FIG. 14 is showing the H-E staining result of kidney membrane of an animal 100 days after being transplanted with PEG grafted pancreatic islet. By comparing those pictures, it is confirmed that lymphocytes infiltration was remarkably low in PEG crafted pancreatic islet.

### Example 9

### Microcapsulation of PEG-Grafted Pancreatic Islet

<9-1> Microcapsulation of PEG-Grafted Pancreatic Islet

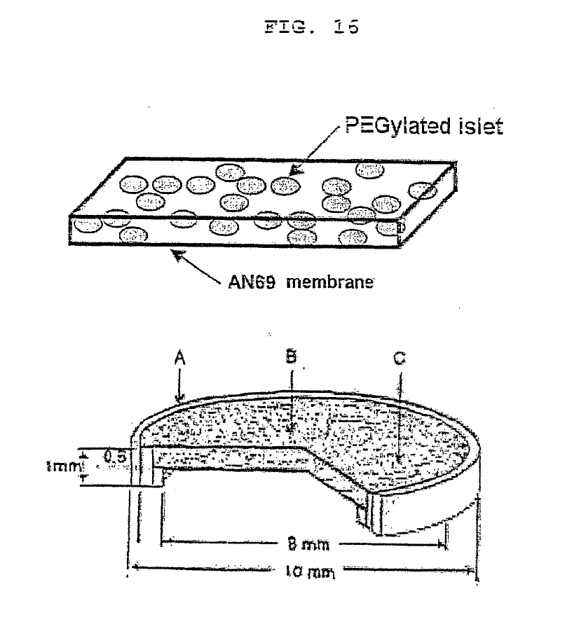

In order to microcapsulate pancreatic islet whose membrane surface was grafted with polymeric PEG, PEG grafted islet in the concentration of 3000/ml was suspended in purified 1.5% (w/v) sodium alginate solution. The above suspension was loaded into 100 mM CaCl₂ solution using 10 ml syringe with air-jet pumping method, resulted in preparation of alginate microcapsule. The microcapsule having PEG grafted islet washed with 50 mM CaCl₂, and then washed again with 25 mM CaCl₂, and finally washed with 150 mM saline solution. Thereafter, the microcapsule was reacted in 0.05% (w/v) poly-L-lysine (2000 Mr) solution for 5 minutes and then washed with 5 nM 2-N-cyclohexylamino ethanesulfonic acid, pH 7.4), followed by washing with 150 mM saline solution for 3 minutes. The microcapsule then was suspended in 0.15% sodium alginate solution for 4-5 minutes, which washed again with 55 mM sodium citrate solution for 4 minutes, resulted in liquefaction of alginate gel inside of the capsule. Lastly, the microcapsule washed with saline solution and washed again with PPMI-1640 culture medium, followed by transferring into the same medium for further culturing (FIG. 15).

<9-2> Transplantation of PEG-Grafted Pancreatic Islet Enveloped with Microcapsule

1000 control pancreatic islet and 2000 microcapsulated PEG-grafted islet were separately transplanted to kidney membranes of diabetic animals with the same method as <4-1> and the changes of their weight and blood-sugar level were observed.

As a result, the animals transplanted with control pancreatic islet without PEG showed increased sugar level up to over 500 mg/dl 2 weeks later, but other animals transplanted with microcapsulated PEG-grafted islet kept normal sugar level and showed normal weight increase as of 100 days after transplantation.

### Example 10

### Macrocapsulation of PEG-Grafted Pancreatic Islet

<10-1> Macrocapsulation of PEG-Grafted Pancreatic Islet

In order to envelop pancreatic islet grafted with polymeric PEG on its membrane surface with macrocapsule, following device was used. Macrocapsulation device consists of membrane supporter which is 10 mm in diameter and 1 mm thick constructed with three rings of polytetrafluoroethylene (PTFE) 20 μm thick AN69 membrane which is used for renal dialysis constructed with 69% acrylonitrile and 31% sodium methally sulfonate, and collagen type I matrix which prevent aggregation of islets. Collagen type I matrix was taken from the Achilles' tendon of a rat tail and dissolved in sterilized acetic acid (1/1000 v/v) solution, after which that solution was centrifuged at 16000×g, 4° C. for 1 hour. Supernatant was obtained therefrom and stored at 4° C. To prepare collagen gel, the above cooled collagen solution, culture base and 0.14 mM sodium bicarbonate solution were mixed with the volume ratio of 7:1:2 each and then cooled the mixture rightly in an ice water bath in order to prevent gelation. The right amount of PEG-grafted pancreatic islet was suspended in the cooled mixture and later, the macrocapsulation device was filled with the suspension using 40 μl pipette for completing macrocapsulation. The device was fixed at 37° C. for 10 minutes, after which the chamber was covered with AN69 membrane (FIG. 16).

<10-2> Transplantation of Macrocapsulated PEG-Grafted Pancreatic Islet

1000 control pancreatic islet and 2000 macrocapsulated PEG-grafted islet were translated onto kidney membranes of diabetic animals with the same method as the above <4-1> and the changes of their weight and blood-sugar level were observed.

As a result, the blood-sugar level of animals transplanted with control pancreatic islet without PEG increased up to over 500 mg/dl 2 weeks later but the blood-sugar level of other animals transplanted with macrocapsulated PEG-grafted pancreatic islet was kept normally and their weight increased normally as of 100 days after transplantation.

### INDUSTRIAL APPLICABILITY

As seen above, surface modification of pancreatic islet membranes by polymeric grafting methods of the present invention can be used to increase the viability of pancreatic islet owing to its power of preventing aggregation of islets by transforming islet surfaces into hydrophilic polymers when being used for microcapsulation or macrocapsulation. More precisely, surface modification of islet membranes by grafting polymeric chains like PEG to the surface of islet glycogen membrane can moderate immune response against islet transplantation and not only prolong the survival and increase the efficiency of islet by supplying enough oxygen and nutrients but also can be very useful for industrial uses by minimizing the whole volume of islet for transplantation.

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended claims.

1. A method for modifying the surface of a pancreatic islet comprising the following steps:
1) carboxylating polyethylene glycol (PEG) via esterification and hydrolysis to result in a modified PEG comprising a carboxy group;
2) activating the carboxy group obtained in step 1) to result in an activated PEG comprising a mixed anhydride; and
3) grafting the activated PEG obtained in step 2) onto the surface of the membrane of the pancreatic islet by chemically binding the activated PEG to a first functional group of the surface.

2. The method for modifying the surface of the pancreatic islet as set forth in claim 1, wherein the PEG is hydrophilic.

3. The method for modifying the surface of the pancreatic islet as set forth in claim 1, wherein the molecular weight of PEG is 100-1,000,000 daltons.

4. The method for modifying the surface of the pancreatic islet as set forth in claim 2, wherein the PEG has a second functional group.

5. The method for modifying the surface of the pancreatic islet as set forth in claim 4, wherein the second functional group is a member selected from the group consisting of an amine group, a hydroxyl group, a carboxyl group, and a sulfone group.

6. The method for modifying the surface of the pancreatic islet as set forth in claim 1, wherein the first functional group of the surface is a member selected from the group consisting of an amine group, a hydroxyl group, a carboxyl group, a thiol group and an azide group.

7. The method for modifying the surface of the pancreatic islet as set forth in claim 1, wherein the step 2) is performed once or more than once.

8. The method for modifying the surface of the pancreatic islet as set forth in claim 1, wherein the method further comprises the following step:
4) after step 3), amplifying the PEG grafted to the surface with a polymer.

9. The method for modifying of the surface of the pancreatic islet as set forth in claim 8, wherein the polymer is a member selected from the group consisting of polyacrylic acid and polyvinyl alcohol.

10. The method for modifying the surface of the pancreatic islet as set forth in claim 1, wherein the PEG in step 1) has one or more second functional groups.

11. The method for modifying the surface of the pancreatic islet as set forth in claim 10, wherein the one or more second functional groups of the PEG selected from the group consisting of a sulfone group and a carboxyl group.
